(19) United States
(12) Patent Application Publication
(10) Pub. No.: US 2026/0363672 A1
(43) Pub. Date: Nov. 19, 2026

(54) ARTIFICIAL INTELLIGENCE (AI) DRIVEN ADAPTIVE SETTINGS AND RESOLUTION

(71) Applicant: GE Precision Healthcare LLC, Waukesha, WI (US)

(72) Inventors: Michael G. Cannon, Haverford, PA (US); Nicolas Poilvert, Denver, CO (US); Jamie Elise Prezioso, Washington, DC (US); Kayla Sue Matcheck, Durham, NC (US); Jeanie Jo Standard, Glendale, WI (US)

(21) Appl. No.: 19/209,473

(22) Filed: May 15, 2025

(51) Int. Cl. A61B 8/00; G16H 30/40; G16H 40/63

(52) U.S. Cl. CPC A61B 8/54; A61B 8/5246; A61B 8/5276; A61B 8/58; G16H 30/40; G16H 40/63

(57) ABSTRACT

Disclosed herein are ultrasound imaging systems which provide an imaging procedure using machine learning systems, which can be used, for example, to assist in acquisition of diagnostic images for assessing a health condition of a target organ of a subject.

### BACKGROUND

Ultrasound imaging is a non-invasive diagnostic modality that is portable and affordable. It does not use ionizing radiation and can be applied in a wide range of medical applications. Certain imaging systems process the ultrasound images prior to submission to machine learning (ML) systems. In some instances, this provides a reduced image quality. In some instances, the ultrasound imaging system and process described herein improves system properties including thermal regulation, image quality, data storage, and battery life described herein. In some instances, the ultrasound imaging system and process described herein are used as a diagnostic procedure involving either a sweep mode or a non-sweep mode. Each mode requires adjusting one or more settings of the ultrasound imaging system by modifying the resolution, fame rate, and/or sweep count. Accordingly, described herein are methods and systems that submit images to the machine learning system to determine acquisition parameters, which may be used to subsequently transform acquisition instructions.

### SUMMARY

In some aspects, described herein, is an ultrasound imaging system configured for conducting an ultrasound imaging procedure, the system comprising: an ultrasound imaging probe; a computing system; and a computer-readable storage medium, storing instructions that, when executed by a processor of the computing system cause the ultrasound imaging system to: (a) obtain a plurality of images using the ultrasound imaging probe, wherein obtaining the plurality of images comprises actuating the ultrasound imaging probe in a non-sweep mode; (b) submit the plurality of images to a machine learning model to obtain one or more acquisition parameters; and (c) transform the one or acquisition parameters to one or more acquisition instructions, wherein the acquisition instructions are provided to the ultrasound imaging system.

In some aspects, described herein, is a method for conducting an ultrasound imaging procedure, the method comprising: (a) obtaining a plurality of images using the ultrasound imaging probe, wherein obtaining the plurality of images comprises actuating the ultrasound imaging probe in a non-sweep mode; (b) submitting the plurality of images to a machine learning model to obtain one or more acquisition parameters; and (c) transforming the one or more acquisition parameters to one or more acquisition instructions, wherein the one or more acquisition instructions are provided to the ultrasound imaging system.

In some aspects, the one or more acquisition instructions comprise one or more instructions for a user of the ultrasound imaging system, and/or one or more updated acquisition parameters for use during a subsequent acquisition.

In some aspects, the one or more acquisition parameters determines one or more user metrics, wherein the one or more user metrics transform one or more movements.

In some aspects, the one or more movements are provided until a target view is reached.

In some aspects, the non-sweep mode directs performance of a calibration procedure comprising providing one or more acquisition instructions to a user of the ultrasound imaging system to conduct one or more movements at an adjusted resolution compared to a default resolution setting.

In some aspects, the non-sweep mode improves a function of a wireless probe or a wired probe, wherein the function is selected from the group: data storage, thermal control, and image quality.

In some aspects, the non-sweep mode instructs the computing system to use 50% or less power compared to a default power setting for mechanical index and/or compared to a default power setting for thermal index.

In some aspects, the one or more acquisition instructions comprise an instruction to acquire a subsequent ultrasound images at an adjusted resolution compared to a default resolution setting of the ultrasound imaging system.

In some aspects, an average resolution mode comprises at least 30 frames per rate.

In some aspects, the one or more movements optimize at least one system property.

In some aspects, the at least one system property is selected from the group consisting of improved thermal control, improved image quality, improved data storage, improved battery life, and a combination thereof.

In some aspects, the target view is determined based at least in part on a diagnostic procedure.

In some aspects, the non-sweep mode has a clinical application in myocardial strain imaging.

In some aspects, the one or more movements change one or more settings of the ultrasound imaging system.

In some aspects, the one or more settings comprises modifying the resolution, frame rate, sweep count, or a combination thereof.

In some aspects, the ultrasound imaging system repeats the non-sweep mode after a sweep at an adjusted resolution compared to a default resolution setting.

In some aspects, the non-sweep mode magnifies the target feature in a target organ.

In some aspects, the target organ comprises a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid.

In some aspects, described herein, is a computer system for conducting an ultrasound imaging procedure, the computer system configured to: (a) obtain a plurality of images using the ultrasound imaging probe, wherein obtaining the plurality of images comprises actuating the ultrasound imaging probe in a non-sweep mode; (b) submit the plurality of images to a machine learning model to obtain one or more acquisition parameters; and (c) transform the one or more acquisition parameters to one or more acquisition instructions, wherein the acquisition instructions are provided to the ultrasound imaging system.

In some aspects, described herein, is an ultrasound imaging system configured to conduct an ultrasound imaging procedure, the system comprising: an ultrasound imaging probe; a computing system; and a computer-readable storage medium storing instructions that, when executed by a processor of the computing system, cause the ultrasound imaging system to: (a) obtain a plurality of images using the ultrasound imaging probe, wherein obtaining the plurality of images comprises actuating the ultrasound imaging probe in a sweep mode; (b) submit the plurality of images to a machine learning model to obtain one or more acquisition parameters; (c) transform the one or more acquisition parameters to one or more acquisition instructions; and (d) provide the one or more acquisition instructions to the ultrasound imaging system.

In some aspects, described herein, is a method for conducting an ultrasound imaging produce, the method comprising: (a) obtaining a plurality of images using an ultrasound imaging probe, wherein obtaining the plurality of images comprises actuating the ultrasound imaging probe in a sweep mode; (b) submitting the plurality of images to a machine learning model to obtain one or more acquisition parameters; (c) transforming the one or more acquisition parameters to one or more acquisition instructions; and (d) providing the one or more acquisition instructions to the ultrasound imaging system.

In some aspects, the one or more acquisition instructions comprise one or more instructions for a user of the ultrasound imaging system, and/or one or more updated acquisition parameters for use during a subsequent acquisition.

In some aspects, the one or more acquisition parameters determines a user metrics, wherein the user metrics transform one or more movements.

In some aspects, the one or more movements are provided until a target view is reached.

In some aspects, the sweep mode instructs the computing system to use 50% or less power compared to a default power setting for mechanical index and/or compared to a default power setting for thermal index.

In some aspects, the sweep mode directs a user to perform a calibration procedure comprising providing one or more acquisition instructions to a user of the ultrasound imaging system to conduct one or more movements at an adjusted resolution compared to a default resolution setting.

In some aspects, the sweep mode improves a function of a wireless probe or a wired probe, wherein the function is selected from the group: data storage, thermal control, and image quality.

In some aspects, the calibration is based at least in part on a target view.

In some aspects, the ultrasound imaging system transforms the one or more acquisition parameters into the one or more acquisition instructions by submitting the plurality of ultrasound images to the machine learning model to obtain one or more user instructions, wherein the one or more user instructions directs a user to perform one or more movements.

In some aspects, the one or more movements comprise one or more adjustments of one or more settings of the ultrasound imaging system.

In some aspects, the one or more settings comprises modifying the resolution, the frame rate, the sweep count, or a combination thereof.

In some aspects, a target view is determined based at least in part on a diagnostic procedure.

In some aspects, the one or more movements comprise a high-resolution sweep.

In some aspects, the high-resolution sweep is directed to be performed after a first sweep.

In some aspects, the ultrasound imaging system repeats the sweep mode in real-time after the high-resolution sweep.

In some aspects, the one or more movements optimizes at least one system property.

In some aspects, the at least one system property is selected from the group consisting of improved thermal control, improved image quality, improved data storage, improved battery life, and a combination thereof.

In some aspects, the sweep mode has a clinical application in obstetrics.

In some aspects, described herein, is a computer system for conducting an ultrasound imaging procedure, the computer system configured to: (a) obtain a plurality of images using the ultrasound imaging probe, wherein obtaining the plurality of images comprises actuating the ultrasound imaging probe in a sweep mode; (b) submit the plurality of images to a machine learning model to obtain one or more user metrics; (c) transform the one or more acquisition parameters to one or more acquisition parameters; and (d) provide the one or more acquisition instructions to the ultrasound imaging system.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative aspects, in which the principles of the invention are utilized, and the accompanying drawings (also “Figure” and “FIG.” herein), of which:

FIG. 1 illustrates an example workflow for directing a non-sweep mode of an ultrasound imaging procedure according to aspects described herein.

FIG. 2 illustrates an example workflow for directing a non-sweep mode of an ultrasound imaging procedure according to aspects described herein.

FIG. 3 illustrates an example workflow for directing a non-sweep mode of an ultrasound imaging procedure according to aspects described herein.

FIG. 4 illustrates an example workflow for directing a non-sweep mode of an ultrasound imaging procedure according to aspects described herein.

FIG. 5 illustrates an example workflow for directing a non-sweep mode of an ultrasound imaging procedure according to aspects described herein.

FIG. 6 illustrates an example workflow for directing a sweep mode of an ultrasound imaging procedure according to aspects described herein.

FIG. 7 illustrates an example workflow for directing a sweep mode of an ultrasound imaging procedure according to aspects described herein.

FIG. 8 illustrates an example workflow for directing a sweep mode of an ultrasound imaging procedure according to aspects described herein.

FIG. 9 shows a computer system that is programmed or otherwise configured to implement methods provided herein.

FIGS. 10A-H illustrates a series of high-resolution diagnostic acquisition sweeps across the abdomen.

FIGS. 11A-C illustrates a series of low-resolution calibration sweeps.

### DETAILED DESCRIPTION

While various aspects of the invention have been shown and described herein, it will be obvious to those skilled in the art that such aspects are provided by way of example only.

Numerous variations, changes, and substitutions may occur to those skilled in the art without departing from the invention. It should be understood that various alternatives to the aspects of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive aspects herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

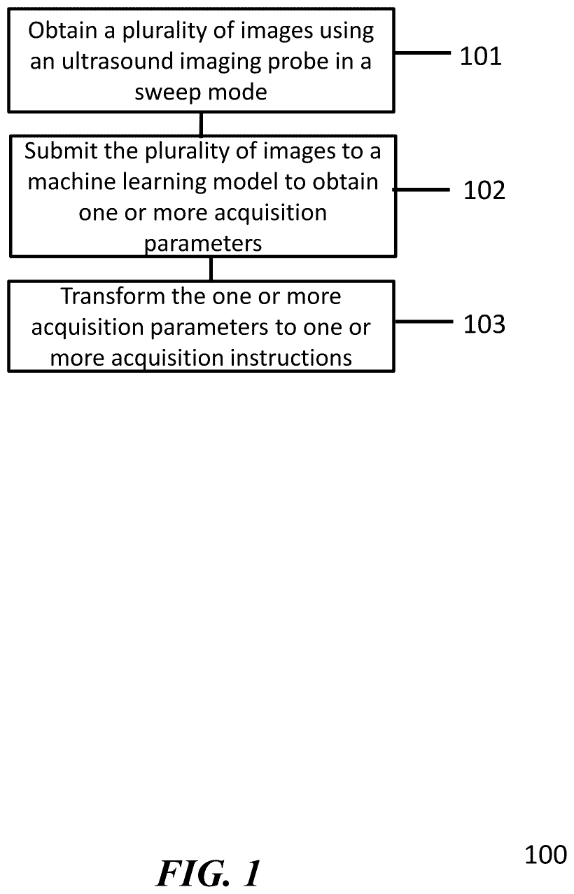

Methods and systems described herein can be implemented according to numerous alternative workflows. For example, the workflow 100 illustrated in FIG. 1 involves: obtaining a plurality of images using an ultrasound imaging probe in a sweep mode 101. The ultrasound imaging system may vary depending on the diagnostic procedure, target organ (e.g., a heart, a liver, a lung, a kidney, a stomach, a small intestine, a large intestine, a pancreas, a brain, a skin, a spleen, an appendix, a bladder, a gallbladder, an eye, an adrenal gland, a gall bladder, a pituitary gland, a component of the male reproductive system, a component of the female reproductive system, a muscle, a fetus, a thyroid), and/or target view.

The workflow described herein involves submitting the plurality of images to a machine learning model to obtain one or more acquisition parameters 102 and transforming the one or more acquisition parameters to one or more acquisition instructions 103. In some embodiments, the patient or subject is an individual seeking treatment. In some embodiments, the patient or subject has a disease or disorder. In some embodiments, the patient or subject is considered healthy and/or performing a routine health check. In some embodiments, the patient or subject is a human.

Acquisition parameters provided herein may be used to assist a user in acquiring subsequent ultrasound images having an improved system property (e.g., thermal control, data storage, image quality, and battery life) and/or an improved clinical or diagnostic quality as described herein. In some embodiments, image quality improvements include reducing pixelation, clutter, speckle, or noise. In some embodiments, the acquisition parameters transform the acquisition instructions by submitting the ultrasound images to a ML model. The ML model may provide user instructions from the acquisition instructions. The one or more acquisition instructions can adjust one or more acquisition parameters. In some embodiments, the one or more acquisition parameters are adjusted automatically. In some embodiments, the one or more acquisition parameters are adjusted manually. In some embodiments, the one or more acquisition instructions adjust one or more settings. In some embodiments, the one or more settings comprise the resolution, frame rate, the sweep count, or a combination thereof. In some embodiments, the one or more settings includes adjusting the position of the probe. In some embodiments, the probe is a wired probe or a wireless probe. The one or more acquisition parameters may determine one or more user metrics. The one or more user metrics may direct a user to perform one or more movements. In some embodiments, the one or more user metrics are performed automatically. In some embodiments, the one or more movements are further directed by the ML model. Sweep mode may comprise a plurality of steps. In some embodiments, sweep mode comprises performing a series of sweeps. A series of sweeps may comprise a plurality of sweeps. In some embodiments, a sweep comprises moving the probe over a target view and/or target organ. A sweep may be performed at an adjusted resolution compared to a default resolution setting. In some embodiments, an adjusted resolution comprises reducing the resolution or increasing the resolution. A sweep may be performed at a reduced resolution compared to a default resolution setting. A sweep may be performed at an increased resolution compared to a default resolution setting. In some embodiments, the decreased resolution in comparison to the default resolution setting comprises about 10%, about 20%, about 30%, about 40%, about 50%, about 60%, about 70%, about 80%, or about 90%. In some embodiments, the increased resolution compared to a default resolution setting comprises about 10%, about 30%, about 50%, about 70%, about 90%, about 110%, about 130%, about 150%, about 170%, about 190%, or about 200%. In some embodiments, the increased resolution comprises the maximum clinically allowable resolution.

The one or more acquisition instructions may direct the user to adjust one or more settings (e.g., position of the probe, frame rate, sweep count, resolution, or any combination thereof). In some embodiments, the user is a novice. In some embodiments, the user is performing a series of blind sweeps where the user is not required to understand or even see the images being created. In some embodiments, the user makes a series of blind sweeps over an area of interest. In some embodiments, the series of blind sweep requires that the user is not aware of the target organ and/or target view. In some embodiments, the ML model analyzes the series of blind sweeps. In some embodiments, the ML model identifies anatomical targets and/or automatically produces interpretation findings or measurements.

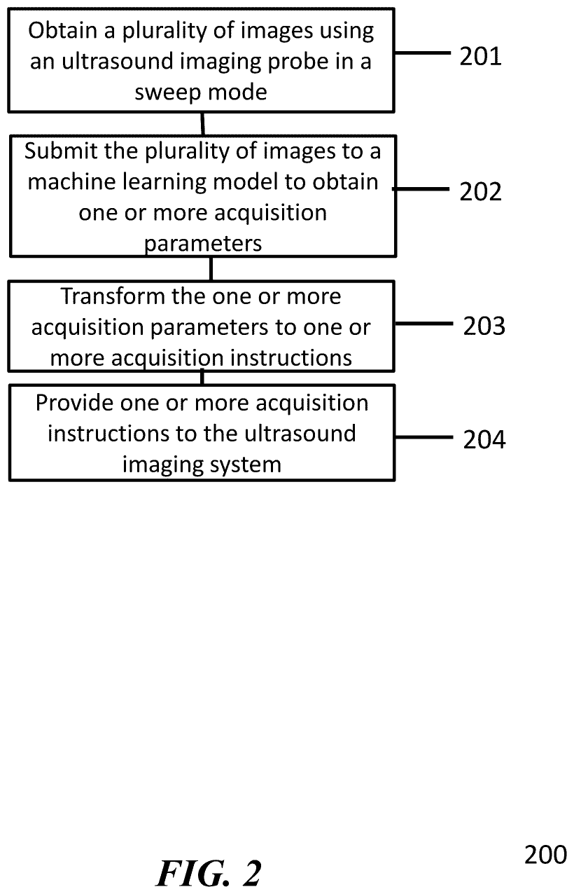

FIG. 2 outlines the workflow described herein 200. The workflow involves: obtaining a plurality of images using an ultrasound imaging probe in a sweep mode 201. The ultrasound imaging system may vary depending on the diagnostic procedure, target organ (e.g., a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid), and/or target view. For example, the ultrasound imaging probe of an ultrasound imaging system (e.g., using a transducer of a two-dimensional ultrasound imaging system such as a linear probe, a curvilinear probe, or a phased array probe) may be a wired probe or a wireless probe. The plurality of images may be submitted to a ML system to obtain one or more acquisition parameters 202. The ML system may transform acquisition parameters to one or more acquisition instructions 203. The acquisition instructions may, for example, provide a user direction on one or more movements. The one or more acquisition instructions may be provided to the ultrasound imaging system 204. The one or more acquisition instructions may generate user instructions. The user instructions may be directed to adjust one or more settings (e.g., modify the position of the probe, resolution, frame rate, sweep count, or a combination thereof). The user instructions may direct the user to repeat an acquisition. The workflow described herein provides improved efficiency of the ultrasound imaging system. In some embodiments, the one or more settings may be adjusted until a target view is reached. The target view may vary depending on the diagnostic procedure and/or clinical application. In some embodiments, the diagnostic procedure and/or clinical application depends on the user's ability to perform a diagnosis. Diagnosis factors may include clarity, presence, and/or structural visibility of target view and/or target structure.

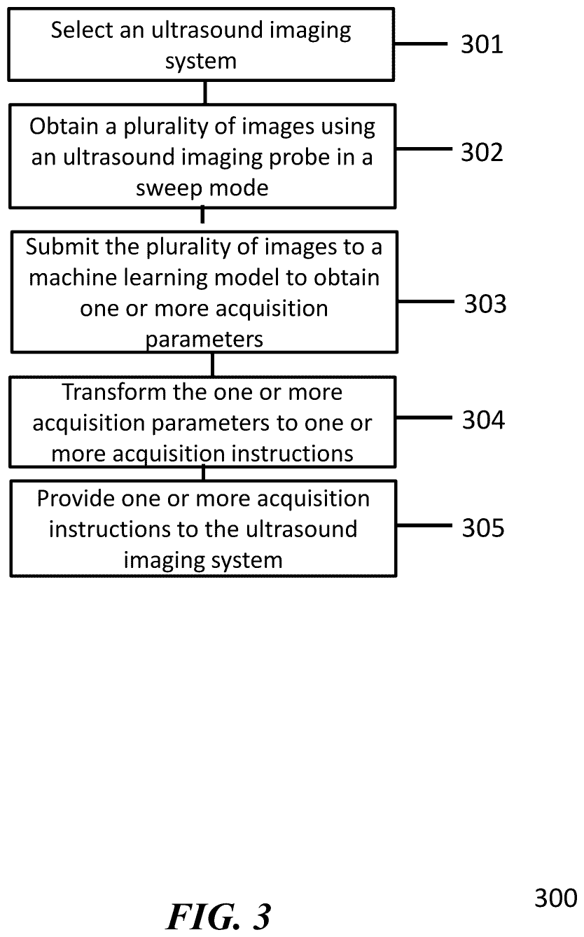

The workflow 300 illustrated in FIG. 3 comprises: selecting an ultrasound imaging system 301. The ultrasound imaging system may vary depending on the diagnostic procedure, target organ (e.g., a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid), and/or target view. For example, the ultrasound imaging probe of an ultrasound imaging system (e.g., using a transducer of a two-dimensional ultrasound imaging system such as a linear probe, a curvilinear probe, or a phased array probe) may be a wired probe or a wireless probe. The plurality of images may be captured in sweep mode using the ultrasound imaging probe 302. Sweep mode may comprise a series of sweeps. A sweep may involve scanning the ultrasound imaging probe over a target organ and/or target view. Sweep mode may comprise a calibration step. The calibration step may comprise a series of preliminary sweeps at an adjusted resolution compared to a default resolution setting. In some embodiments, the adjusted resolution comprises a reduced resolution. In some embodiments, the adjusted resolution comprises an increased resolution. The ML model may adjust the one or more acquisition parameters based on the calibration step. In some embodiments, the one or more acquisition parameters are automatically updated via the ML model. In some embodiments, the one or more acquisition parameters are manually updated by the user. In some embodiments, the ML model adjusts one or more acquisition parameters based on a plurality of images captured after the calibration step. The one or more acquisition parameters may direct one or more acquisition instructions. The acquisition instructions may direct the user to adjust one or more settings via user instructions (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle.

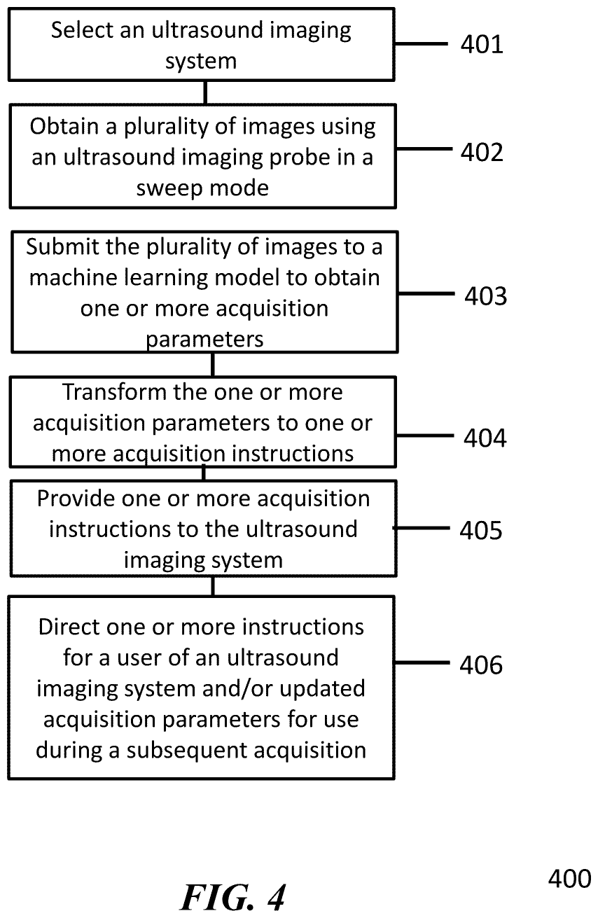

The workflow 400 as described herein is illustrated in FIG. 4. FIG. 4 involves: selecting an ultrasound imaging system 401. The ultrasound imaging system may vary depending on the diagnostic procedure, target organ (e.g., a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid), and/or target view. The ultrasound imaging system may obtain a plurality of images using an ultrasound imaging probe in a sweep mode 402. The plurality of images can be captured, for example, using a wired or wireless ultrasound imaging probe (e.g., using a transducer of a two-dimensional ultrasound imaging system such as a linear probe, a curvilinear probe, or a phased array probe). The plurality of images may be submitted to a ML model to obtain one or more acquisition parameters 403. Sweep mode may comprise a series of sweeps. A sweep may involve scanning the ultrasound imaging probe over a target organ and/or target view. Sweep mode may comprise a calibration step. The calibration step may comprise a series of preliminary sweeps at an adjusted resolution compared to a default resolution setting. In some embodiments, the adjusted resolution may comprise increasing the resolution compared to the default resolution setting. In some embodiments, the adjusted resolution may comprise reducing the resolution compared to the default resolution setting. The ML model may adjust the one or more acquisition parameters based on the calibration step. In some embodiments, the calibration step is performed at a reduced resolution compared to the default resolution setting. In some embodiments, the calibration step is performed at an increased resolution compared to the default resolution setting. In some embodiments, the ML model may adjust one or more acquisition parameters after the calibration step. In some embodiments, the ML model may automatically adjust one or more acquisition instructions and direct one or more acquisition parameters. The ML model may transform the one or more acquisition parameters to one or more acquisition instructions 404. In some embodiments, the one or more acquisition parameters determines one or more user metrics. In some embodiments, the one or more user metrics transform one or more movements. The ML model may provide one more acquisition instructions to the ultrasound imaging system 405. The ML model may direct one or more instructions for a user of an ultrasound imaging system and/or updated acquisition parameters for a user during a subsequent acquisition 406. In some embodiments, the acquisition parameters are manually updated by the user. The acquisition instructions may direct the user to adjust one or more settings via user instructions (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle.

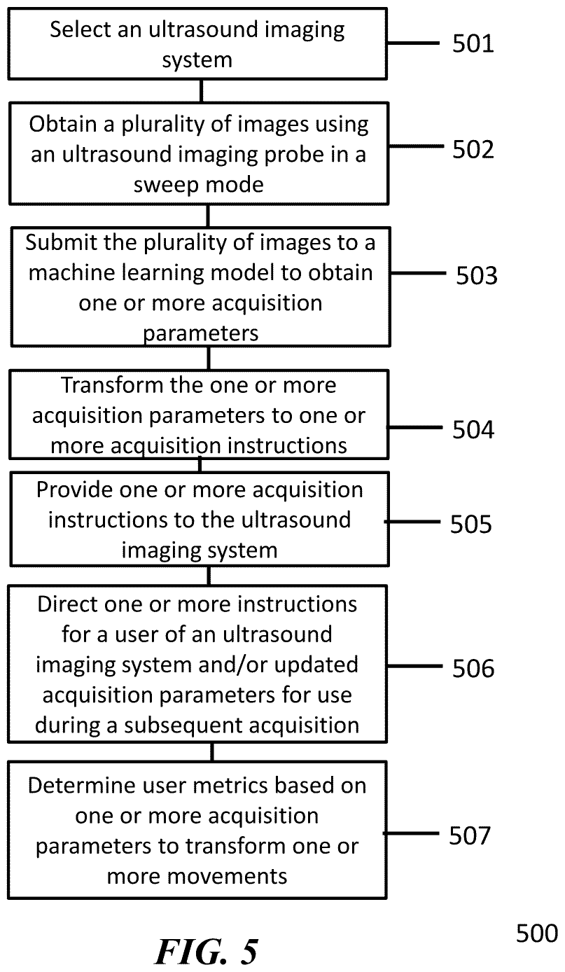

The workflow as described herein 500 is illustrated in FIG. 5. FIG. 5 comprises: selecting an ultrasound imaging system 501. The ultrasound imaging system may vary depending on the diagnostic procedure, target organ (e.g., a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid), and/or target view. The ultrasound imaging system may obtain a plurality of images using an ultrasound imaging probe in a sweep mode 502. For example, the plurality of images can be captured using a probe. In some embodiments, the probe comprises a wireless or a wired ultrasound imaging probe (e.g., using a transducer of a two-dimensional ultrasound imaging system such as a linear probe, a curvilinear probe, or a phased array probe). The plurality of images may be submitted to a ML model to obtain one or more acquisition parameter 503. The ML model may transform the one or more acquisition parameters to one or more acquisition instructions 504. The ML model may provide the one or more acquisition instructions to the ultrasound imaging system 505. The ML model may direct one or more instructions for a user of an ultrasound imaging system and/or updated acquisition parameters for user during a subsequent acquisition 506. In some embodiments, the ML model automatically updates the acquisition parameters. In some embodiments, the acquisition parameters are updated manually by the user. The one or more acquisition parameters are used to determine user metrics. The user metrics can further transform one or more movements 507. The one or more movements may improve one or more settings via user instructions (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle.

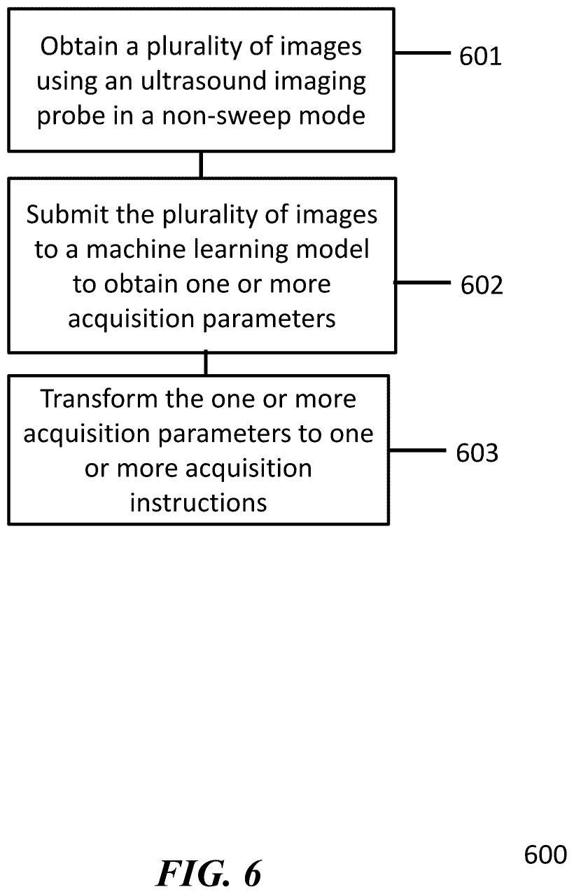

The workflow as described herein 600 is illustrated in FIG. 6. FIG. 6 comprises: obtaining a plurality of images using an ultrasound imaging probe in a non-sweep mode 601 and submitting the plurality of images to a ML model to obtain one or more acquisition parameters 602. For example, the plurality of images can be captured using a wireless or wired ultrasound imaging probe (e.g., using a transducer of a two-dimensional ultrasound imaging system such as a linear probe, a curvilinear probe, or a phased array probe). In some embodiments, the patient or subject is an individual seeking treatment. In some embodiments, the patient or subject has a disease or disorder. In some embodiments, the patient or subject is considered healthy and/or performing a routine health check. In some embodiments, the patient or subject is a human. The plurality of images in both first mode and second may be captured, for example, using an imaging probe of an ultrasound imaging system (e.g., using a transducer of a two-dimensional ultrasound imaging system such as a linear probe, a curvilinear probe, or a phased array probe). The ML model may transform the one more acquisition parameter into one or more acquisition instructions 603. The acquisition instructions may be provided to a user to adjust one or more settings (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle. In some embodiments, the user is medical staff. In some embodiments, the user is a skilled clinician. In some embodiments, the user is not a novice.

Non-sweep mode may comprise a plurality of steps. In some embodiments, the plurality of steps comprise a series of sweeps. In some embodiments, the sweep comprises scanning the probe over a target view and/or target organ in order to diagnose a patient. In some embodiments, the patient has an abnormality or pathology. In some embodiments, the patient does not have an abnormality or pathology. In some embodiments, the non-sweep mode is used for a diagnosis. In some embodiments, the non-sweep mode is used in a clinical application. In some embodiments the operator is skilled in the performance of ultrasound imaging and recognizes anatomical structures. In some embodiments the operator is not skilled in the performance of ultrasound imaging and does not recognize anatomical structures. In some embodiments, the set of sweeps is performed as a preliminary step, also known as the calibration step. In some embodiments, the calibration step is performed at an adjusted resolution compared to a default resolution setting. In some embodiments, the adjusted resolution comprises an increased or decreased resolution compared to the default resolution setting. In some embodiments, the ML model automatically adjusts one or more acquisition instructions and directs the one or more acquisition parameters. In some embodiments, the subsequent acquisitions are at an at a reduced resolution compared to a default resolution setting. In some embodiments, the subsequent acquisitions are at an increased resolution compared to a default resolution setting. A non-sweep may involve scanning the ultrasound imaging probe over a target organ and/or target view. In some embodiments, the decreased resolution in comparison to the default resolution setting comprises about 10%, about 20%, about 30%, about 40%, about 50%, about 60%, about 70%, about 80%, or about 90%. In some embodiments, the increased resolution compared to a default resolution setting comprises about 10%, about 30%, about 50%, about 70%, about 90%, about 110%, about 130%, about 150%, about 170%, about 190%, or about 200%. In some embodiments, the increased resolution comprises the maximum clinically allowable resolution. In some embodiments, the user is analyzing the series of sweeps in conjunction with the ML model to identify anatomical targets and/or automatically produce interpretation findings or measurements. In some embodiments, the ML model automatically adjusts the one or more acquisition instructions and directs the one or more acquisition parameters. In some embodiments, the ML model adjusts one or more acquisition parameters based on subsequent images captured after the calibration step. In some embodiments, the ML model adjusts one or more acquisition parameters throughout the process of capturing images using the ultrasound imaging system. In some embodiments, the non-sweep mode has a clinical application.

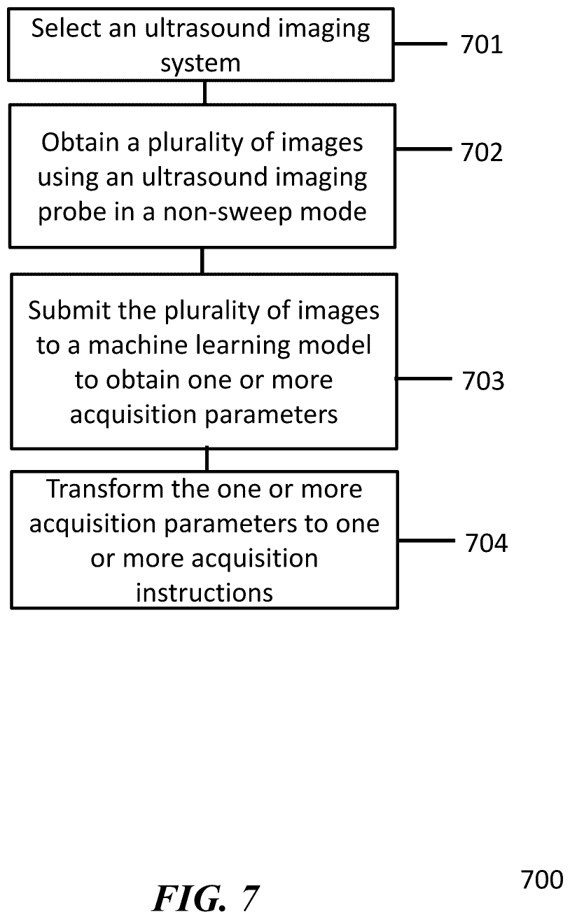

The example workflow 700 illustrated in FIG. 7 comprises: selecting an ultrasound imaging system 701. The ultrasound imaging system may vary depending on the diagnostic procedure, target organ, and/or target view (e.g., obstetrics and heart). The ultrasound imaging system may obtain a plurality of images using an ultrasound imaging probe in a non-sweep mode 702. For example, the plurality of images can be captured using a wireless or wired ultrasound imaging probe (e.g., using a transducer of a two-dimensional ultrasound imaging system such as a linear probe, a curvilinear probe, or a phased array probe). The plurality of images may be submitted to a ML model to obtain one or more acquisition parameters 703. The one or more acquisition parameters may be transformed to one or more user instructions 704. The acquisition instructions may be provided to a user to adjust one or more settings (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle. In some embodiments, the non-sweep mode comprises the user analyzing the series of sweeps in conjunction with the ML model to identify a target view and/or validate a diagnostic procedure.

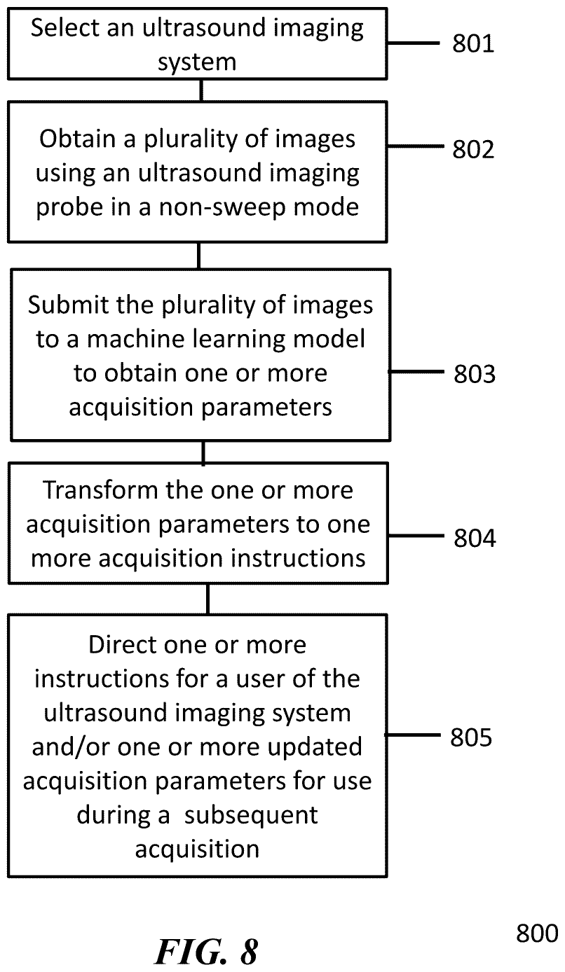

The example workflow 800, as illustrated in FIG. 8 comprises: selecting an ultrasound imaging system 801. The ultrasound imaging system may vary depending on the diagnostic procedure, target organ, and/or target view (e.g., obstetrics and heart). The ultrasound imaging system may obtain a plurality of images using an ultrasound imaging probe in a non-sweep mode 802. For example, the plurality of images can be captured using a wireless or wired ultrasound imaging probe (e.g., using a transducer of a two-dimensional ultrasound imaging system such as a linear probe, a curvilinear probe, or a phased array probe). The plurality of images may be submitted to a ML model to obtain one or more acquisition parameters 803 The one or more acquisition parameters may be transformed to one or more user instructions 804. The ML model may direct one or more instructions for a user of the ultrasound imaging system and/or one or more updated acquisition parameters for use during a subsequent acquisition. The processed images may further be submitted to a ML model 805. The one or more acquisition instructions may be provided to a user to adjust one or more settings (e.g., position of the probe, frame rate, sweep count, resolution, or any combination thereof). In some cases, the user instructions may improve one or more system properties (e.g., improved thermal control, improved image quality, improved data storage, and improved battery life). In some embodiments, improved image quality comprises improved contrast and clarity; and reduce pixelation, noise, clutter, or speckle. In some embodiments, the non-sweep mode comprises the user analyzing the series of sweeps in conjunction with the ML model to identify a target view and/or validate a diagnostic procedure. The one or more acquisition parameters may be continually updated in view of new data submitted to the ML model.

### Example Images

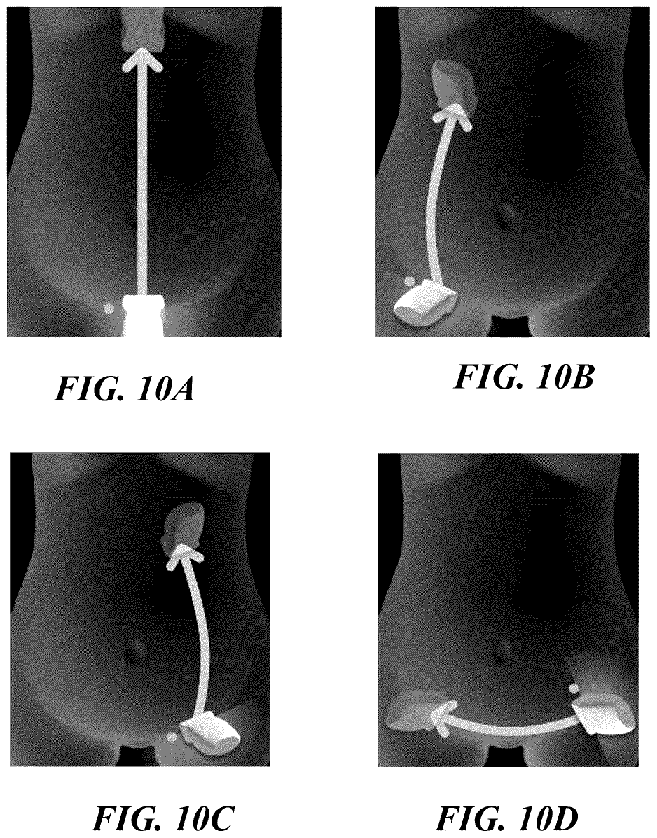

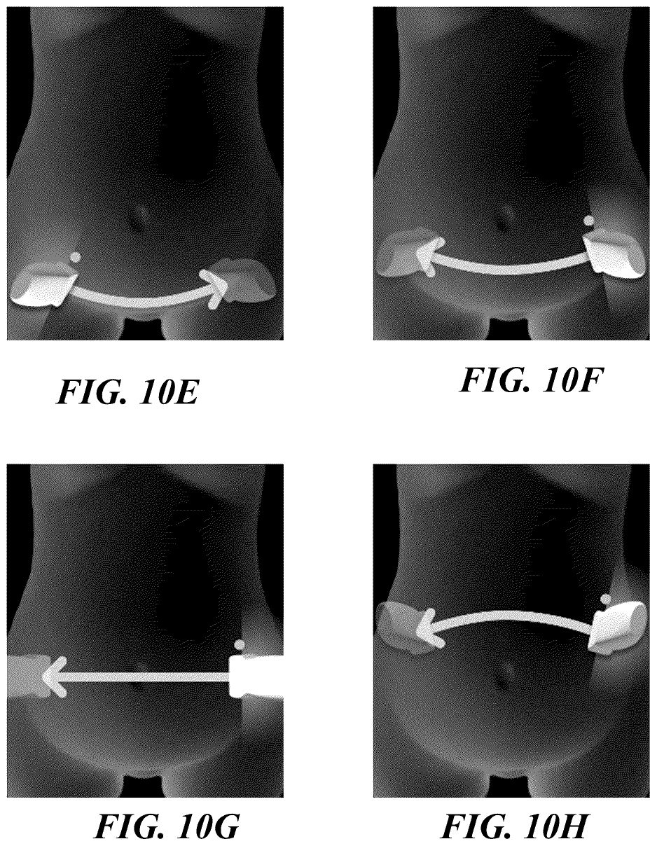

FIGS. 10A-H provide an example of diagnostic acquisition sweeps over the abdomen. In some embodiments, the diagnostic acquisition sweeps are known as blind sweeps. In some embodiments, the diagnostic acquisition sweeps are utilized in obstetrics. The images the series of sweeps directed by the ML model in response to the calibration step. In some embodiments, the images are captured in an increased resolution compared to the default resolution setting. In some embodiments, the images are captured at a reduced resolution compared to the default resolution setting. In FIGS. 10A-C, the sweeps are performed in an up and down motion. In FIGS. 10C-H, the sweeps are performed in a motion from left to right or right to left.

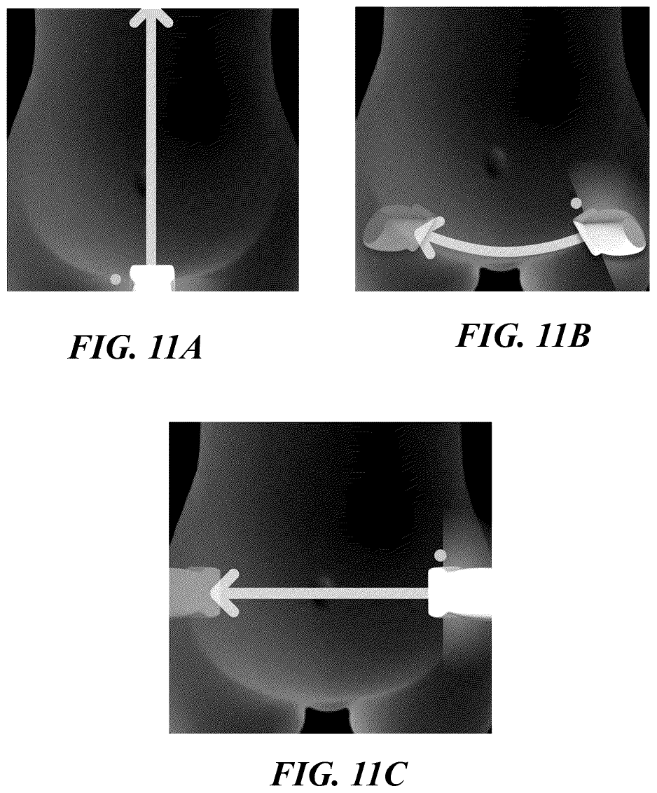

FIGS. 11A-C provide an example of the images captured using the low-resolution calibration sweep. FIG. 11A depicts a sweep in an up and down motion while FIGS. 11B-C depict a sweep in a motion from left to right or right to left.

### Adaptive Settings

A particular challenge in ultrasound medical imaging concerns data processing capacity limits reducing diagnostic quality and performance. In some cases, battery-powered (e.g., wireless) probes are limited in image quality because of the need to miniaturize the ultrasound electronics in the probe. In order to manage thermal control and extended runtime, these probes may need to operate with settings that result in lower image quality. For example, wireless probes may reduce the scan angle, lower frame rates, reduce scanline, beam density, reduce axial sampling rates, and number of focal zones. In some cases, wireless probes are unable to provide higher levels of image quality than that required for advanced diagnostic interpretation or measurements. The invention described herein is better equipped at managing one or more system properties and can consequently prepare images with improved quality and more effective diagnostic functions.

In some cases, the examination is performed by a non-expert. The non-expert may attempt to acquire images using a “blind sweep.” In some cases, the “blind sweep” results in the user not being required to understand or even see the images being created. Instead, the user can be directed to make multiple sweeps over an area of interest by a Machine Learning (ML) algorithm. The ML algorithm can analyze the frames of the blind sweeps. In some cases, the ML algorithm identifies anatomical targets and/or automatically produces interpretation findings or measurements. Multiple blind sweeps can produce large files, which can be a challenge for low-cost, portable, or handheld ultrasound imaging systems. In particular, multiple blind sweeps can require large data size.

Moreover, large data size can be difficult to upload from remote or rural areas with low wireless bandwidth. The invention described herein can use the ML algorithm to direct multiple blind sweeps to ensure better function of low-cost, portable, or handheld ultrasound imaging systems.

Furthermore, the invention described herein allows the diagnostic capability of ultrasound imaging systems to be extended to underserved areas.

The invention described herein may improve at least one system property. In some embodiments, the at least one system property comprises improved thermal control, improved image quality, improved data storage, improved battery life, or any combination thereof. As used herein, an image quality (e.g., diagnostic quality or clinical quality) may be used to refer to one or more aspects of the quality of an image. In some aspects, image quality is in reference to an image that can be viewed by a trained expert. In some aspects, image quality is in reference to an image in which the targets are displayed in a clear and well-defined manner, for example, where extraneous noise or clutter is minimal, the grayscale display shows subtle variations of tissue type and texture, frame rates are high, providing accurate depiction of tissue, aeration or lack thereof, or blood flow movement, borders between tissue types or blood flow and vessel or other structures are well resolved, ultrasound artifacts such as grating and side lobes are minimized, acoustic noise is absent, places to make measurements in the image are obvious and distinct, or any combination thereof depending on the nature of the ultrasound exam. In some aspects, image quality is in reference to an image that contains the necessary anatomical targets to represent a standard diagnostic view. In some aspects, image quality is in reference to an image in which a diseased condition, abnormality, or pathology is well visualized. For example, medical images may be labeled by healthcare professionals according to whether they are considered to have a well visualized diseased condition, abnormality, or pathology, and then used to train a machine learning algorithm to differentiate between images based on image quality. In some aspects, image quality means that some combination of these aforementioned characteristics is present.

Accordingly, disclosed herein are platforms, systems, and methods comprising one or more algorithms for evaluating ultrasound images to provide acquisition parameters. In some aspects, the acquisition parameters are provided in real time. In some embodiments, the acquisition parameters provide acquisition instructions to the user. The acquisition instructions may further be updated in real-time in response to new data.

In some embodiments, the acquisition instructions comprise one or more movements. The one or more movements may include changing settings, moving the probe, and/or moving the patient or subject position. In some embodiments, the acquisition instructions may direct the one or more movements to enhance the image quality (e.g., improve clarity and contrast; and reduce pixelation, speckle, clutter, and noise). In some embodiments, the improvement in image quality improves the visualization of a target structural feature or a target organ of a subject. In some embodiments, the acquisition instructions are applied to a diagnostic procedure. In some embodiments, the acquisition instructions can be continually updated in response to new data.

In some embodiments, the ML model assess the quality of the acquisition parameter and determine whether additional images are required. In some embodiments, the acquisition parameter directs the user to change the position of the probe. In some embodiments, the acquisition parameter directs the user to capture replacement images.

### Machine Learning Algorithms

Disclosed herein are platforms, systems, and methods that provide ultrasound image classification using machine learning algorithm(s). The ML and AI system described herein can be used to calculate user metrics. In some embodiments, the user metrics can provide a contemporaneous image quality score and/or guided movements.

In particular, in some aspects, the machine learning algorithms include deep learning neural networks configured for evaluating ultrasound images. The algorithms can include one or more of a positioning algorithm, a scoring algorithm, a probe guidance algorithm, and an intrinsic image quality algorithm. The positioning algorithm can include one or more neural networks that estimate probe positioning relative to an ideal anatomical view or perspective and/or a distance or deviation of a current probe position from an ideal probe position. The intrinsic image quality algorithm may determine that intrinsic image quality is below a threshold based in part on a determination by a positioning algorithm that one or more images have been acquired at a probe position expected to obtain a clinical quality image.

The development of each machine learning algorithm spans three phases: (1) dataset creation and curation, (2) algorithm training, and (3) adapting design elements necessary for product performance and useability. The dataset used for training the algorithm can be generated by obtaining the user metrics from a plurality of images captured under first and/or second mode. Each algorithm then can be modified using data collected from the plurality of images, which can include one or more different target organs and/or one or more different views of a given target organ.

A machine learning model can comprise a supervised, semi-supervised, unsupervised, or self-supervised machine learning model. In some cases, the one or more ML approaches perform classification or clustering of the MS data. In some examples, the machine learning approach comprises a classical machine learning method, such as, but not limited to, support vector machine (SVM) (e.g., one-class SVM, linear or radial kernels, etc.), K-nearest neighbor (KNN), isolation forest, random forest, logistic regression, AdaBoost classifier, extra trees classifier, extreme gradient boosting, gaussian process classifier, gradient boosting classifier, light gradient boosting, linear discriminant analysis, naïve Bayes, quadratic discriminant analysis, ridge classifier, or any combination thereof. In some examples, the machine learning approach comprises a deep leaning method (e.g., deep neural network (DNN)), such as, but not limited to a fully-connected network, convolutional neural network (CNN) (e.g., one-class CNN), recurrent neural network (RNN), transformer, graph neural network (GNN), convolutional graph neural network (CGNN), multi-level perceptron (MLP), or any combination thereof.

In some aspects, a classical ML method comprises one or more algorithms that learns from existing observations (i.e., known features) to predict outputs. In some aspects, the one or more algorithms perform clustering of data. In some examples, the classical ML algorithms for clustering comprise K-means clustering, mean-shift clustering, density-based spatial clustering of applications with noise (DBSCAN), expectation-maximization (EM) clustering (e.g., using Gaussian mixture models (GMM)), agglomerative hierarchical clustering, or any combination thereof. In some aspects, the one or more algorithms perform classification of data. In some examples, the classical ML algorithms for classification comprise logistic regression, naïve Bayes, KNN, random forest, isolation forest, decision trees, gradient boosting, support vector machine (SVM), or any combination thereof. In some examples, the SVM comprises a one-class SMV or a multi-class SVM.

In some aspects, the deep learning method comprises one or more algorithms that learns by extracting new features to predict outputs. In some aspects, the deep learning method comprises one or more layers. In some aspects, the deep learning method comprises a neural network (e.g., DNN comprising more than one layer). Neural networks generally comprise connected nodes in a network, which can perform functions, such as transforming or translating input data. In some aspects, the output from a given node is passed on as input to another node. The nodes in the network generally comprise input units in an input layer, hidden units in one or more hidden layers, output units in an output layer, or a combination thereof. In some aspects, an input node is connected to one or more hidden units. In some aspects, one or more hidden units is connected to an output unit. The nodes can generally take in input through the input units and generate an output from the output units using an activation function. In some aspects, the input or output comprises a tensor, a matrix, a vector, an array, or a scalar. In some aspects, the activation function is a Rectified Linear Unit (ReLU) activation function, a sigmoid activation function, a hyperbolic tangent activation function, or a Softmax activation function.

The connections between nodes further comprise weights for adjusting input data to a given node (i.e., to activate input data or deactivate input data). In some aspects, the weights are learned by the neural network. In some aspects, the neural network is trained to learn weights using gradient-based optimizations. In some aspects, the gradient-based optimization comprises one or more loss functions. In some aspects, the gradient-based optimization is gradient descent, conjugate gradient descent, stochastic gradient descent, or any variation thereof (e.g., adaptive moment estimation (Adam)). In some further aspects, the gradient in the gradient-based optimization is computed using backpropagation. In some aspects, the nodes are organized into graphs to generate a network (e.g., graph neural networks). In some aspects, the nodes are organized into one or more layers to generate a network (e.g., feed forward neural networks, convolutional neural networks (CNNs), recurrent neural networks (RNNs), etc.). In some aspects, the CNN comprises a one-class CNN or a multi-class CNN.

In some aspects, the neural network comprises one or more recurrent layers. In some aspects, the one or more recurrent layers are one or more long short-term memory (LSTM) layers or gated recurrent units (GRUs). In some aspects, the one or more recurrent layers perform sequential data classification and clustering in which the data ordering is considered (e.g., time series data). In such aspects, future predictions are made by the one or more recurrent layers according to the sequence of past events. In some aspects, the recurrent layer retains or “remembers” important information, while selectively “forgets” what is not essential to the classification.

In some aspects, the neural network comprises one or more convolutional layers. In some aspects, the input and the output are a tensor representing variables or attributes in a data set (e.g., features), which may be referred to as a feature map (or activation map). In such aspects, the one or more convolutional layers are referred to as a feature extraction phase. In some aspects, the convolutions are one-dimensional (1D) convolutions, two dimensional (2D) convolutions, three dimensional (3D) convolutions, or any combination thereof. In further aspects, the convolutions are 1D transpose convolutions, 2D transpose convolutions, 3D transpose convolutions, or any combination thereof.

The layers in a neural network can further comprise one or more pooling layers before or after a convolutional layer. In some aspects, the one or more pooling layers reduces the dimensionality of a feature map using filters that summarize regions of a matrix. In some aspects, this down samples the number of outputs, and thus reduces the parameters and computational resources needed for the neural network. In some aspects, the one or more pooling layers comprises max pooling, min pooling, average pooling, global pooling, norm pooling, or a combination thereof. In some aspects, max pooling reduces the dimensionality of the data by taking only the maximums values in the region of the matrix. In some aspects, this helps capture the most significant one or more features. In some aspects, the one or more pooling layers is one dimensional (1D), two dimensional (2D), three dimensional (3D), or any combination thereof.

The neural network can further comprise of one or more flattening layers, which can flatten the input to be passed on to the next layer. In some aspects, an input (e.g., feature map) is flattened by reducing the input to a one-dimensional array. In some aspects, the flattened inputs can be used to output a classification of an object. In some aspects, the classification comprises a binary classification or multi-class classification of visual data (e.g., images, videos, etc.) or non-visual data (e.g., measurements, audio, text, etc.). In some aspects, the classification comprises binary classification of an image (e.g., contrast needed or contrast not needed). In some aspects, the classification comprises multi-class classification of a text (e.g., identifying hand-written digits)). In some aspects, the classification comprises binary classification of a measurement. In some examples, the binary classification of a measurement comprises a classification of a system's performance using the physical measurements described herein (e.g., normal or abnormal, normal or anormal).

The neural networks can further comprise of one or more dropout layers. In some aspects, the dropout layers are used during training of the neural network (e.g., to perform binary or multi-class classifications). In some aspects, the one or more dropout layers randomly set some weights as 0 (e.g., about 10%, 20%, 30%, 40%, 50%, 60%, 70%, 80% of weights). In some aspects, the setting some weights as 0 also sets the corresponding elements in the feature map as 0. In some aspects, the one or more dropout layers can be used to avoid the neural network from overfitting.

The neural network can further comprise one or more dense layers, which comprises a fully connected network. In some aspects, information is passed through a fully connected network to generate a predicted classification of an object. In some aspects, the error associated with the predicted classification of the object is also calculated. In some aspects, the error is backpropagated to improve the prediction. In some aspects, the one or more dense layers comprises a Softmax activation function. In some aspects, the Softmax activation function converts a vector of numbers to a vector of probabilities. In some aspects, these probabilities are subsequently used in classifications, such as classifications of one or a plurality of image features comprised in one or a plurality of ultrasound images of a lung of a subject.

Computer systems

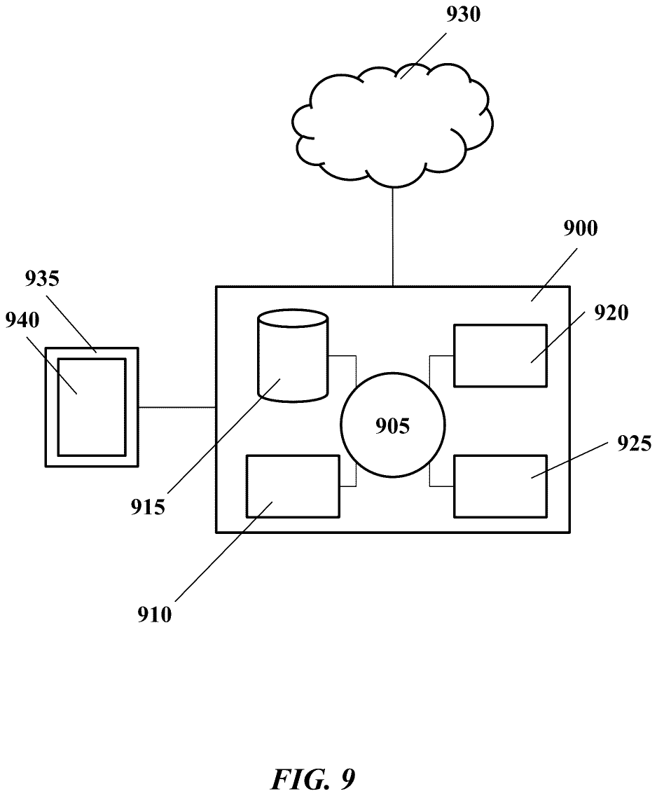

The present disclosure provides computer systems that are programmed to implement methods of the disclosure. FIG. 9 shows a computer system 900 that is programmed or otherwise configured to assess whether an ultrasound enhancing agent is expected to improve image quality according to any of the methods described herein. The computer system 900 can regulate various aspects of the present disclosure. The computer system 900 can be an electronic device of a user or a computer system that is remotely located with respect to the electronic device. The electronic device can be a mobile electronic device.

The computer system 900 includes a central processing unit (CPU, also “processor” and “computer processor” herein) 905, which can be a single core or multi core processor, or a plurality of processors for parallel processing. The computer system 900 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 900 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 900, can implement a peer-to-peer network, which may enable devices coupled to the computer system 900 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 900 can be included in the circuit. In some cases, the circuit is an application specific integrated circuit (ASIC).

The storage unit 915 can store files, such as drivers, libraries, and saved programs. The storage unit 915 can store user data, e.g., user preferences and user programs. The computer system 900 in some cases can include one or more additional data storage units that are external to the computer system 900, such as located on a remote server that is in communication with the computer system 900 through an intranet or the Internet.

The computer system 900 can communicate with one or more remote computer systems through the network 930. For instance, the computer system 900 can communicate with a remote computer system of a user (e.g., a professional sonographer or an untrained technician). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 900 via the network 930.

Methods as described herein can be implemented by way of machine (e.g., computer processor) executable code stored on an electronic storage location of the computer system 900, such as, for example, on the memory 910 or electronic storage unit 915. The machine executable or machine-readable code can be provided in the form of software. During use, the code can be executed by the processor 905. In some cases, the code can be retrieved from the storage unit 915 and stored on the memory 910 for ready access by the processor 1005. In some situations, the electronic storage unit 915 can be precluded, and machine-executable instructions are stored on memory 910.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 900, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system.

Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 900 can include or be in communication with an electronic display 1035 that comprises a user interface (UI) 940 for providing, for example, providing a user with an indication of whether or not an ultrasound enhancing agent is needed to improve an intrinsic image quality. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, be configured to perform any of the methods described herein.

### Ultrasound Annotations

In some embodiments, the plurality of images are captured using an ultrasound imaging probe. In some embodiments, the plurality of images are not captured using an ultrasound imaging probe. In some embodiments, the plurality of images are actuated in sweep mode. In some embodiments, sweep mode directs a series of blind scans. In some embodiments, the plurality of images captured by the blind scans are submitted to a machine learning (ML) model. In some embodiments, the ML model obtains one or more acquisition parameters. In some embodiments, the one or more acquisition parameters direct one or more acquisition instructions. In some embodiments, the one or more acquisition instructions direct one or more movements.

In some embodiments, the ML model does not provide one or more acquisition parameters. In some embodiments, the one or more acquisition parameters do not provide one or more acquisition instructions. In some embodiments, the acquisition instructions do not provide one or more movements.

In some embodiments, the ML model does not identify a target view. In some embodiments, the target view is not based on the anatomical features. The anatomical features may include but are not limited to a heart, a liver, a lung, a kidney, a stomach, a small intestine, a large intestine, a pancreas, a brain, a skin, a spleen, an appendix, a bladder, a gallbladder, an eye, an adrenal gland, a bone, a mouth, a pituitary gland, a component of the male reproductive system, a component of the female reproductive system, a muscle, a nose or a thyroid. In some embodiments, the target view is not based on the structural features. The structural features may include but are not limited to the anatomical boundaries, shapes, and relative positions of different tissues and organs. In some embodiments, the target view is not based on a specific target of the anatomical feature. In some embodiments, the specific targets are on the left or right side of the anatomical feature. In some embodiments, the specific targets are up or down on the anatomical feature.

In some embodiments, the plurality of images are not processed using a transducer. The transducer may be a convex/curvilinear transducer, a linear transducer, or a phased/sector transducer.

In some embodiments, the ML model does not improve at least one system property. In some embodiments, the ML model does not improve image quality. In some embodiments, the ML model does not improve thermal control. In some embodiments, the ML model does not improve data storage. In some embodiments, the ML model does not improve battery life. In some embodiments, the ML model does not adjust the resolution. In some embodiments, adjusting the resolution comprises reducing the resolution compared to the default power setting. In some embodiments, adjusting the resolution comprises increasing the resolution compared to the default power setting. In some embodiments, adjusting the resolution does not reduce the default power setting for mechanical index and/or compared to a default power setting for thermal index.

In some embodiments, the plurality of images are analyzed by a clinician. The clinician may be a technical expert. The clinician may be utilizing the ultrasound imaging system for a diagnostic procedure. In some embodiments, the diagnostic procedure has a clinical application. In some embodiments, the clinical application is obstetrics. In some embodiments, the clinical application is myocardial strain imaging. In some embodiments the diagnostic procedure has a target organ or a feature of a target organ. In some embodiments, the target organ is the heart, liver, thyroid, or any combination thereof.

In some embodiments, the plurality of images are captured during a calibration step. The calibration step is performed at a reduced resolution compared to the default resolution setting. In some embodiments, the calibration step is performed at an increased resolution compared to the default resolution setting. In some embodiments, the resolution is sufficient and the clinician makes no changes. The clinician may review the plurality of images after the calibration step. The clinician may analyze the plurality of images after the calibration step. The clinician may direct the ML model to alter one or more acquisition parameters. In some embodiments, the one or more acquisition parameters provide one or more acquisition instructions to the user. In some embodiments, the clinician may alter the one or more acquisition parameters in accordance with one or more system settings. The clinician may direct the one or more acquisition parameters to increase the frame rate compared to the default frame rate settings. In some embodiments, the clinician directs the one or more acquisition parameters to decrease the frame rate compared to the default frame rate settings. In some embodiments, the frame rate is sufficient and the clinician makes no changes. In some embodiments, the average resolution mode comprises at least 30 frames per rate. In some embodiments, the average resolution mode is at least about 10 frames per rate, about 20 frames per rate, about 30 frames per rate, about 40 frames per rate, about 50 frames per rate, or about 60 frames per rate.

The clinician may direct the one or more acquisition parameters to increase the sweep count compared the default sweep count settings. In some embodiments, the clinician directs the one or more acquisition parameters to decrease the sweep count compared to the default sweep count settings. In some embodiments, the sweep count is sufficient and the clinician makes no changes. The clinician may direct the one or more acquisition parameters to alter one or more settings including the frame rate, the sweep count, the resolution, or any combination thereof.

The clinician may direct the one or more acquisition parameters to improve one or more system settings. In some embodiments, the clinician may direct the ML model to improve thermal control. In some embodiments, the clinician may direct the one or more acquisition parameters to improve battery life. In some embodiments, the clinician may direct the one or more acquisition parameters to improve image quality. In some embodiments, improved image quality comprises improved contrast and clarity; and reduce pixelation, noise, clutter, or speckle.

In some embodiments, the clinician continuously reviews the plurality of images and updates the ML model as needed.

In some embodiments, the one or more acquisition parameters direct user metrics. In some embodiments, the user metrics direct one or movements. The movements may comprise moving the position of the probe. In some embodiments, the user metrics direct one or movements until a target view is reached. In some embodiments, the user may alter the one or more movements in real-time. In some embodiments, the movements may comprise altering one or more settings. In some embodiments, the one or more settings comprise modifying the resolution, frame rate, sweep count, or a combination thereof. In some embodiments, the one or more movements optimize at least one system property. In some embodiments, the at least one system property comprises thermal control, improved image quality, improved data storage, and improved battery life.

In some embodiments, the plurality of images are not processed for a target view. In some embodiments, the plurality of images are not processed to increase clarity and/or contrast. In some embodiments, the plurality of images are not processed to retain, retune, and/or retest. In some embodiments, the plurality of images are not processed for an internal scan. In some embodiments, the plurality of images are not processed for an external scan. In some embodiments, the plurality of images are not processed for a diagnostic procedure. In some embodiments, the plurality of images are not processed in real-time. In some embodiments, the plurality of images are not processed under a ML system. In some embodiments, the plurality of images are not processed using AI. In some embodiments, the plurality of images are not processed to magnify depth, angle, and/or line density. In some embodiments, the plurality of images are not processed for a diagnostic procedure. In some embodiments, the plurality of images are not processed for an electrocardiogram (i.e., EKG), abdominal scan, obstetric ultrasound, Doppler ultrasound, transabdominal ultrasound, transrectal ultrasound, thyroid scan, transvaginal scan, testicular scan, carotid ultrasound, venosus ultrasound, 3D ultrasound, and/or a 4D ultrasound.

### EXAMPLES

### Example 1

### Machine Learning Models for Guidance of Imaging Procedures in a Sweep Mode

Described herein is imaging system or processing a plurality of images 500 (FIG. 5). In some instances, an ultrasound imaging system is selected 501. In some instances, the plurality of images are obtained using an ultrasound imaging probe in a sweep mode 502. In some instances, the plurality of images are submitted to the machine learning (ML) model. The plurality of images may be analyzed by the ML model. The ML model obtains one or more acquisition parameters 503. Furthermore, the ML model transforms the one or more acquisition parameter into one or more acquisition instructions 504. In some instances, the one or more acquisition instructions are provided to the ultrasound imaging system 505. The acquisition instructions are provided to a user of an ultrasound imaging system. In some instances, the ML model may update the one or more acquisition parameters for use in a subsequent acquisition 506. In some instances, the user metrics are determined based on the one or more acquisition parameters to transform one or more movements. The movements may comprise moving the position of the probe and/or adjusting one or more settings. In some instances, the one or more settings comprise adjusting the resolution, frame rate, sweep rate, or a combination thereof. In some instances, the ultrasound imaging system described herein provides improved system properties, including but not limited to improved image quality, thermal regulation, and data storage. The ultrasound imaging system described herein, in some instances, provides improved image quality associated with reduced pixelation, clutter, and noise and increased clarity and contrast.

The imaging process for the ultrasound imaging system or process described herein encompasses a front end and a back end. The front end includes the transmit circuit control, transducer, beam former, and RF processing. The back end includes the first and second mode, scan conversion, Machine Learning (ML) and Artificial Intelligence (AI) processing, image enhancement processing, and image display and storage. The ultrasound imaging system or process comprises a computer and a probe. The probe may be a wireless probe. The probe can integrate the front-end processes including the transmit circuit control, transducer, and the beamforming. The wireless probe can perform all the functions related to transmitting, receiving, and beamforming in the probe housing. Scan conversion may also be performed in the probe. If scan conversion is software-based, it may also be done on a tablet or phone which the probe is connected to.

The user is directed to capture a calibration sweep. In some embodiments, the calibration sweep is captured at an increased resolution compared to a default resolution setting. In some embodiments, the calibration step is captured at a decreased resolution compared to a default resolution setting. In some instances, the ML provides one or more acquisition instructions based on the one or more acquisition parameters. In some instances, the one or more acquisition instructions are manually updated by the user. In some instances, the user is not a skilled technician. The ML model may automatically update and direct the one or more acquisition instructions. In some instances, the ML model automatically updates the one or more acquisition parameters after the calibration step.

In some instances, the ML model automatically adjusts one or more settings. In some instances, the one or more settings comprise adjusting the resolution, frame rate, sweep rate, or a combination thereof. In some instances, the one or more acquisition instructions comprise adjusting the resolution compared to a default resolution setting. In some instances, adjusting comprises increasing the resolution or reducing the resolution. In some instances, the one or more acquisition instructions comprise an increased resolution compared to a default resolution setting. In some instances, the one or more acquisition instructions comprise a reduced resolution compared to a default resolution setting. In some instances, the one or more acquisition instructions increase the frame rate compared to a default frame rate setting. In some instances, the acquisition instructions decrease the frame rate compared to a default frame rate setting. In some instances, the one or more acquisition instructions increase the sweep rate compared to a default sweep rate setting. In some instances, the one or more acquisition instructions decrease the sweep rate compared to a default sweep rate setting.

### Example 2

### Machine Learning Models for Guidance of Imaging Procedures in a Non-Sweep Mode

Described herein is imaging system or processing a plurality of images 800 (FIG. 8). In some instances, an ultrasound imaging system is selected 801. In some instances, the plurality of images are obtained using an ultrasound imaging probe in a non-sweep mode 802. In some instances, the plurality of images are submitted to the machine learning (ML) model. The plurality of images may be analyzed by the ML model. The ML model obtains one or more acquisition parameters 803. Furthermore, the ML model transforms the one or more acquisition parameter into one or more acquisition instructions 804. In some instances, the one or more acquisition instructions are provided to the ultrasound imaging system 805. The acquisition instructions are provided to a user of an ultrasound imaging system. In some instances, the ML model may update the one or more acquisition parameters for use in a subsequent acquisition. In some instances, the one or more acquisition instructions may direct one or more movements. In some instances, the ML model automatically updates the one or more acquisition instructions and directs the one or more acquisition parameters. In some instances, the one or more acquisition instructions are manually updated by the user. The one or more movements may comprise moving the position of the probe and/or adjusting one or more settings. In some instances, the one or more settings comprise adjusting the resolution, frame rate, sweep rate, or a combination thereof. In some instances, the ultrasound imaging system described herein provides improved system properties, including but not limited to improved image quality, thermal regulation, and data storage. The ultrasound imaging system described herein, in some instances, provides improved image quality associated with reduced pixelation, clutter, and noise and increased clarity and contrast.

The imaging process for the ultrasound imaging system or process described herein encompasses a front end and a back end. The front end includes the transmit circuit control, transducer, beam former, and RF processing. The back end includes the first and second mode, scan conversion, Machine Learning (ML) and Artificial Intelligence (AI) processing, image enhancement processing, and image display and storage. The ultrasound imaging system or process comprises a computer and a probe. The probe may be a wireless probe. The probe can integrate the front-end processes including the transmit circuit control, transducer, and the beamforming. The wireless probe can perform all the functions related to transmitting, receiving, and beamforming in the probe housing. Scan conversion may also be performed in the probe. If scan conversion is software-based, it may also be done on a tablet or phone which the probe is connected to.

The user is directed to capture a calibration sweep. In some embodiments, the calibration sweep is captured at an adjusted resolution compared to a default resolution setting. In some embodiments, the adjusted resolution is at a reduced resolution compared to the default resolution setting. In some embodiments, the adjusted resolution is at an increased resolution compared to a default resolution setting. In some instances, the ML provides one or more acquisition instructions based on the one or more acquisition parameters. In some instances, the user is a skilled technician. The user may respond to the acquisition instructions and adjust one or more settings. In some instances, the one or more settings comprise adjusting the resolution, frame rate, sweep rate, or a combination thereof.

The user may be directed to rotate, tilt, and/or slide the ultrasound transducer probe. In some instances, the one or more acquisition instructions are automatically updated by the ML model. In some instances, the ML model automatically updates the one or more acquisition parameters. In some instances, the one or more acquisition instructions adjust the resolution compared to a default resolution setting. In some instances, adjusting the resolution comprises reducing or increasing the resolution. In some instances, the one or more acquisition instructions reduce the resolution compared to a default resolution setting. In some instances, the one or more acquisition instructions increase the resolution compared to a default resolution setting. In some instances, the one or more acquisition instructions increase the frame rate compared to a default frame rate setting. In some instances, the one or more acquisition instructions decrease the frame rate compared to a default frame rate setting. In some instances, the one or more acquisition instructions increase the sweep rate compared to a default sweep rate setting. In some instances, the one or more acquisition instructions decrease the sweep rate compared to a default sweep rate setting.

1. An ultrasound imaging system configured to conduct an ultrasound imaging procedure, the ultrasound imaging system comprising:
an ultrasound imaging probe;
a computing system; and
a computer-readable storage medium storing instructions that, when executed by a processor of the computing system, cause the ultrasound imaging system to:
obtain a first plurality of images using the ultrasound imaging probe at a reduced resolution compared to a default resolution setting during a first sweep;
submit the first plurality of images to a machine learning model to obtain one or more acquisition parameters;
transform the one or more acquisition parameters to one or more acquisition instructions including a first instruction to automatically update one or more settings of the ultrasound imaging system for a second sweep and a second instruction that instructs a user of the ultrasound imaging system to position the ultrasound imaging probe in accordance with a probe position estimated by the machine learning model for the second sweep;
automatically update the one or more settings of the ultrasound imaging system based on the first instruction;
display guidance information that guides the user of the ultrasound imaging system to position the ultrasound imaging probe in accordance with the probe position estimated by the machine learning model based on the second instruction; and
obtain a second plurality of images using the ultrasound imaging probe during the second sweep based on automatically updating the one or more settings of the ultrasound imaging system and displaying the guidance information.

2. A method for conducting an ultrasound imaging produce, the method comprising:
obtaining a first plurality of images using an ultrasound imaging probe at a reduced resolution compared to a default resolution setting during a first sweep;
submitting the first plurality of images to a machine learning model to obtain one or more acquisition parameters;
transforming the one or more acquisition parameters to one or more acquisition instructions including a first instruction to automatically update one or more settings of an ultrasound imaging system for a second sweep and a second instruction that instructs a user of the ultrasound imaging system to position the ultrasound imaging probe in accordance with a probe position estimated by the machine learning model for the second sweep;
automatically updating the one or more settings of the ultrasound imaging system based on the first instruction;
displaying guidance information that guides the user of the ultrasound imaging system to position the ultrasound imaging probe in accordance with the probe position estimated by the machine learning model based on the second instruction; and
obtaining a second plurality of images using the ultrasound imaging probe during the second sweep based on automatically updating the one or more settings of the ultrasound imaging system and displaying the guidance information.

3. (canceled)

4. The ultrasound imaging system of claim 2, wherein the one or more acquisition parameters determines a user metrics, wherein the user metrics transform one or more movements.

5. The ultrasound imaging system of claim 4, wherein the one or more movements are provided until a target view is reached.

6. The ultrasound imaging system of claim 1, wherein the first sweep uses 50% or less power compared to a default power setting for mechanical index and/or compared to a default power setting for thermal index.

7. (canceled)

8. The ultrasound imaging system of claim 1, wherein the sweep mode improves a function of a wireless probe or a wired probe, wherein the function is selected from the group: data storage, thermal control, and image quality.

9. The ultrasound imaging system of claim 1, wherein the first sweep is performed in accordance with a calibration procedure that is based at least in part on a target view.

10. (canceled)

11. (canceled)

12. The ultrasound imaging system of claim 1, wherein the one or more settings resolution, a frame rate, a sweep count, or a combination thereof.

13. The ultrasound imaging system of claim 12, wherein a target view is determined based at least in part on a diagnostic procedure.

14. The ultrasound imaging system of claim 11, wherein the second sweep is performed at a greater resolution than the reduced resolution of the first sweep.

15. (canceled)

16. (canceled)

17. (canceled)

18. (canceled)

19. (canceled)

20. A computer system for conducting an ultrasound imaging procedure, the computer system configured to:
obtain a first plurality of images using an ultrasound imaging probe at a reduced resolution compared to a default resolution setting during a first sweep;
submit the first plurality of images to a machine learning model to obtain one or more acquisition parameters;
transform the one or more acquisition parameters to one or more acquisition instructions including a first instruction that automatically updates one or more settings of an ultrasound imaging system for a second sweep and a second instruction that instructs a user of the ultrasound imaging system to position the ultrasound imaging probe in accordance with a probe position estimated by the machine learning model for the second sweep;
automatically update the one or more settings of the ultrasound imaging system based on the first instruction;
display guidance information that guides the user of the ultrasound imaging system to position the ultrasound imaging probe in accordance with the probe position estimated by the machine learning model based on the second instruction; and
obtain a second plurality of images using the ultrasound imaging probe during the second sweep based on automatically updating the one or more settings of the ultrasound imaging system and displaying the guidance information.
